(19) United States
(12) Patent Application Publication
(10) Pub. No.: US 2024/0117980 A1
(43) Pub. Date: Apr. 11, 2024

(54) AIR TREATMENT SYSTEM

(71) Applicant: ARURA BLUE LLC, Sacramento, CA (US)

(72) Inventors: James Albert Gavney, JR., Sunnyvale, CA (US); Jesse James Graham, Portland, OR (US); Blane Goettle, Golden River, CA (US)

(73) Assignee: ARURA BLUE LLC, Sacramento, CA (US)

(21) Appl. No.: 18/445,541

(22) Filed: Oct. 4, 2023

Related U.S. Application Data
(60) Provisional application No. 63/475,025, filed on Oct. 5, 2022.
(60) Provisional application No. 63/475,095, filed on Oct. 12, 2022.

(51) Int. Cl. F24F 8/22; F24F 11/74

(52) U.S. Cl. CPC F24F 8/22; F24F 11/74; F24F 2120/10

(57) ABSTRACT

An air treatment system includes germicidal air circulation unit with a germicidal chamber with disinfection stages or channels each having UVC LED light engines therein. The air treatment system includes an intake port, an exhaust port and a circulation fan that pushes or pulls air into and through the germicidal chamber. The germicidal chamber can include baffle structures and can contain a metal-oxide material and/or a metal-sulfide material. The air treatment system includes a control unit and sensor that regulates air flow provided by the circulation fan and/or the fluence or power levels of the UVC LED light engines.

### RELATED APPLICATION

This application claims priority under 35 U.S.C. § 119(e) from the U.S. provisional patent application Ser. No. 63/475,025, filed on Oct. 5, 2023, and titled “GERMICIDAL AND LIGHTING SYSTEM WITH LINEAR AIR CIRCULATION UNITS” and the U.S. provisional patent application Ser. No. 63/475,095, filed on Oct. 12, 2023, and titled “DECENTRALIZED GERMICIDAL AND LIGHTING SYSTEM”. The U.S. provisional patent application Ser. No. 63/475,025 and Ser. No. 63/475,095 are both hereby incorporated by reference.

### FIELD OF THE INVENTION

The invention relates to air treatment systems. More particularly, the present invention relates to air treatment systems with germicidal air circulation chambers.

### BACKGROUND OF THE INVENTION

Germicidal fixtures produce some amount of short-wave ultraviolet (UVC) light that can break of disrupt DNA base pairing, causing formation of pyrimidine dimers, and leading to the inactivation of bacteria, viruses, and protozoa. Germicidal fixtures are used in water disinfecting systems. Germicidal fixtures can include low-pressure mercury lamps, high-pressure mercury lamps, LEDs or combinations thereof.

Germicidal fixtures are used primarily in two different ways. Germicidal fixtures are used to disinfect surfaces, such as workplace surfaces, tools and the like. Germicidal fixtures are also used to disinfect air. Because air-borne pathogens appear to be the most powerful mechanism for spreading viruses, such as Covid-19, air disinfection germicidal fixtures can be an important tool for providing safe indoor spaces.

Ultraviolet (UV) light in the presence of an appropriate catalyst can, in addition to reducing air-bourne pathogens, can also break-down or decompose VOCs (Volatile Organic Compounds) in a process that is referred to as Photo-catalytic Degradation.

### SUMMARY OF THE INVENTION

The present invention is directed to an air treatment system that includes air circulation germicidal units to disinfect air in an occupied indoor space or room. The air circulation germicidal units are preferably mounted in overhead locations within the indoor space or room, such as on or within a ceiling and/or on or within upper wall areas. The germicidal system preferably unitizes multiple air circulation germicidal units that are distributed throughout the indoor space or room. The air circulation germicidal units operate collectively to disinfect the air within the indoor space or room and operate based on detected or measured air quality and/or detected or measures occupancy levels within the indoor space or room.

A germicidal air circulation unit of the present invention includes a germicidal chamber, a circulation fan and a UVC light engine. The UVC light engine is isolated within the germicidal camber to prevent or eliminate UVC radiation leakage and exposure to occupants of the indoor space or room where the germicidal air circulation unit is stationed or installed. In operation, the circulation fan draws air into the germicidal camber through and inlet port, the air is then exposed to radiation from the UVC light engine within the germicidal chamber and the treated air is redistributed into the room through an exhaust port in a continuous cycle.

The germicidal air circulation units can also includes light units with light engines for providing lighting to the indoor space or room. The lighting units are separate from the germicidal air circulation unit or integrated with the germicidal air circulation unit, but are preferably independently operable, such that the germicidal air circulation units and the lighting units can be controlled independently. The UVC light engine in the germicidal air circulation unit is preferably a LED UVC light engine and the light engine in the lighting unit is preferably a white LED light engine.

The air treatment system, with or without the lighting units have controls that can include occupancy sensors, for turning on and off the germicidal air circulation units and/or lighting units in rooms or spaces where the air treatment system is stationed or installed. The air treatment system can also include a CO₂ sensors for determining occupancy levels or air quality levels of rooms or spaces where the air treatment system is stationed or installed and can be used to adjusting circulation rates and/or UVC fluence levels of the germicidal air circulation units.

In further embodiments of the invention the occupancy sensors determines when there is more than one occupants, the number of occupants within a space or room and/or the spacing of occupants within the space or room and controls the air flow, UVC LED fluence or power levels of germicidal air circulation units based on the measured number of occupants and/or spacing of the occupants within the space or room. For example, individual germicidal air circulation units can automatically shut off or turn on when there is only a single occupant within the space and individual germicidal air circulation units can automatically power down when occupants are sufficiently spaced apart.

The air treatment system of the present invention preferable broadcasts and audio signal when the germicidal air circulation units are operating and provides a visual representation of a predicated level of air disinfection that has been provided by germicidal air circulation units at any given time while the air treatment system is in operation.

In accordance with an embodiments of the invention an air treatment system includes suspended linear germicidal air circulation units that can provide down lighting and that are suspended from a ceiling by a cable mounting features or are mounted on a wall with the appropriate hardware. Linear germicidal air circulation units includes germicidal chambers with channels or tracks that have interior walls that are UVC reflective. The interior walls of the channels of tracks can be for example, coated or made from a UVC reflective materials, such as polished Aluminum, Zinc Oxide and/or Titanium Oxide.

In further embodiments of the invention germicidal chambers of the germicidal air circulation units can includes a catalatic material that can assist in a process of breaking-down or decomposing VOCs (Volatile Organic Compounds) in a process that is referred to as Photo-catalytic Degradation. Catalatic materials for Photo-catalytic Degradation include metal-oxides or metal-sulfides that can be doped with a carbon-based nano material, such as graphene.

Where the germicidal chambers are coated with, made from or contain a metal-oxide and/or a metal sulfide based material the air treatment system can also include a VOC sensor that measures VOC levels in the indoor space or room and a control unit that regulates the operation of the germicidal air circulation units based on the measured VOC levels. Suitable metal-oxides and metal-sulfides include, but are not limited to, Zinc Oxide (ZnO), Titanium Oxide (TiO₂), Tungsten Oxide (WO₃), Vanadium Oxide (V₂O₅), Zinc Sulfide (ZnS), and Cadmium Sulfide (CdS) and combinations thereof.

### BRIEF DESCRIPTION OF THE DRAWINGS

FIG. 1 shows a schematic representation am air treatment system with a germicidal air circulation unit and controls for treating air-bourne pathogens, in accordance with the embodiments of the invention.

FIGS. 2A-B illustrates a schematic lay-out of a de-localized or de-centralize upper air treatment system, in accordance with the embodiments of the invention.

FIG. 3A shows a schematic representation canister-style air treatment fixture, in accordance with the embodiments of the invention.

FIGS. 3B-C show cross-sectional schematic representations of germicidal chamber configurations with UVC LED light engines and baffle structures, in accordance with the embodiments of the invention.

FIGS. 4A-C show views of an air treatment fixture, in accordance with the embodiments of the invention.

FIGS. 5A-F show views of a pendent-style air treatment fixture with a germicidal air circulation unit having bi-directional sets of reflective raceways or germicidal cambers, in accordance with the embodiments of the invention.

FIG. 6 shows a schematic representation of an elongated germicidal air circulation unit with a baffle structure, in accordance with the embodiments of the invention.

FIGS. 7A-B shows cross-sectional views of a condensed germicidal air circulation unit with stacked disinfection stages and baffle strictures, in accordance with the embodiments of the invention.

### DETAILED DESCRIPTION OF THE INVENTION

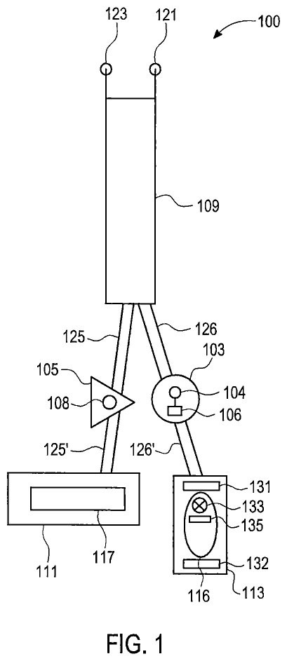

FIG. 1 shows a schematic representations of an air treatment system 100 with germicidal air circulation unit 113 and control unit 103 for disinfecting pathogens in air of an indoor space or room. The air treatment system 100 includes a power source 109, such a driver circuit for connecting to line voltage contacts 121 and 123 and for powering the air treatment system 100.

The germicidal air circulation unit 113 includes an enclosed germinal chamber 116. Within the enclosed germinal chamber 116, there is a circulation fan 133 and a UVC light engine 135, that is preferably a UVC LED light engine. The circulation fan 133 draws the air into the enclosed germicidal camber 116 through an inlet port 132 where the air is exposed to radiation from the UVC light engine 135 and the treated air is then redistributed onto a space or room through an exhaust port 131.

The control unit 103 is connected to the germicidal air circulation unit 113 through the appropriate electrical connections 126 and 126′ and can include an occupancy sensor 104 and a CO₂ sensor 106. The occupancy sensor 103 can turn on and off the germicidal air circulation unit 113 and the CO₂ sensor 106 can control the operating speed of the circulation fan 133 and/or the fluence level or power level of the UVC light engine 135 based on the detected level of CO₂ in the room where the air treatment system 100 is stationed or installed.

The germicidal and lighting system 100 can also includes a lighting unit 111. The lighting unit 111 includes a light engine 117, that is preferably an LED light engine. The lighting unit 111 is powered by the power source 109, or a separate power source, through the appropriate electrical connections 125 and 125′. The lighting unit 111 can also be connected to a separate control unit 105 that includes, for example, an occupancy sensor 108 for turning on and off the light engine 117 based on detected occupancy within the space or room.

In addition to the CO₂ sensor 106 or alternatively to the CO₂ sensor 106, the control unit 103 can includes a VOC sensor that monitors airborne Volatile Organic Compounds within the space or room. The control unit 103 will then control the air flow provided by the fan 133 and/or the fluence or power levels of the UVC LED light engine 135 based levels of VOCs detected.

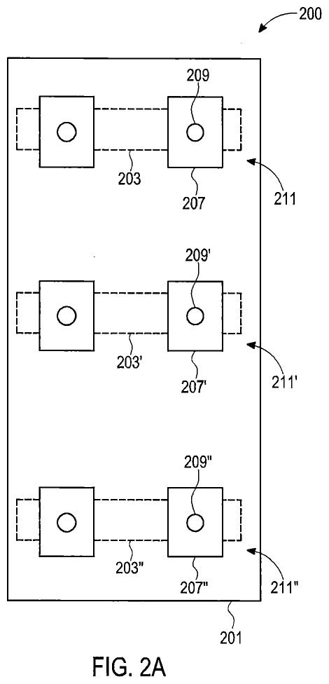

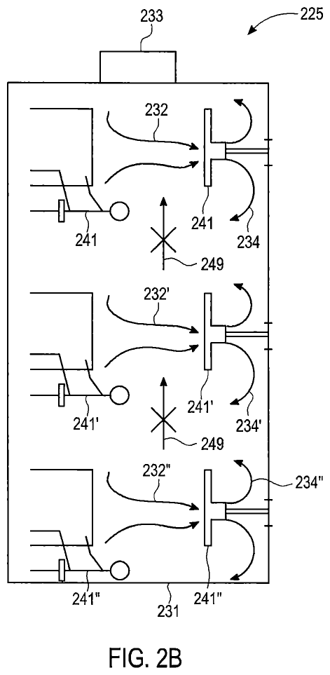

FIG. 2A shows an schematic top view representation 200 of an indoor space or room 201 with a de-centralized or de-localized air treatment system distributed through different zones 211/211′/211″ of the indoor space or room 201. Each of the different zones 211/211′/211″ includes one or more germicidal fixtures 207/207′/207″ that each include a germicidal air circulation unit 209/209′/209″, respectively. The germicidal fixtures 207/207′/207″ are located in overhead positions above, tables, desks, seating areas or in any other suitable overhead location 203/203′/203″ within each of the different zones 211/211′/211″ of the indoor space or room 201.

In operation, each of the germicidal fixtures 207/207′/207″ can operate collectively or independently through a control unit 103 (FIG. 1) based on levels of occupants, CO₂ and or VOCs detected in each of the different zones 211/211′/211″. Each of the germicidal air circulation units 209/209′/209″ has a circulation fan 133, a UVC light engine 135, an inlet port 132 and an exhaust port 131 and operated as described above with reference (FIG. 1). Each of the germicidal fixtures 207/207′/207″ can also include a lighting unit 111 (FIG. 1).

FIG. 2B shows a side perspective schematic representation of an indoor space or room 231 with a de-centralized or de-localized air treatment system 225 that distributed through different zones of the indoor space or room 231, such as described with reference to FIG. 2A. Each of the different zones includes one or more air treatment fixtures 241, 241′ and 241″ that each include a germicidal air circulation unit, such as described above and below. The air treatment fixture 241, 241′ and 241″ are located in overhead locations above, tables, desks, seating areas where occupants 241, 241′ and 241″ commonly reside within each of the different zones of the indoor space or room 231. In operation, air is drawing upward from each of the different zones, as indicated by the arrow of the 232, 232′ and 232″ and into the germicidal air circulation units of the air treatment fixture 241/241′/241″ where the air is treated with UVC light and expelled or redistributed within the space or room 231, as indicated by the arrows 234, 234′ and 234″.

The de-centralized or de-localized air treatment system 225 can also includes a central control unit 233. The central control unit 233 allows for manual control of each of the air treatment fixtures 241, 241′ and 241″ and displays information about the de-centralized or de-localized air treatment system 225 including, but not limited to, predicted air quality within the space or room 231, operating conditions of each of the air treatment fixtures 241, 241′ and 241″ and any potential operational failures of each of the air treatment fixtures 241, 241′ and 241″.

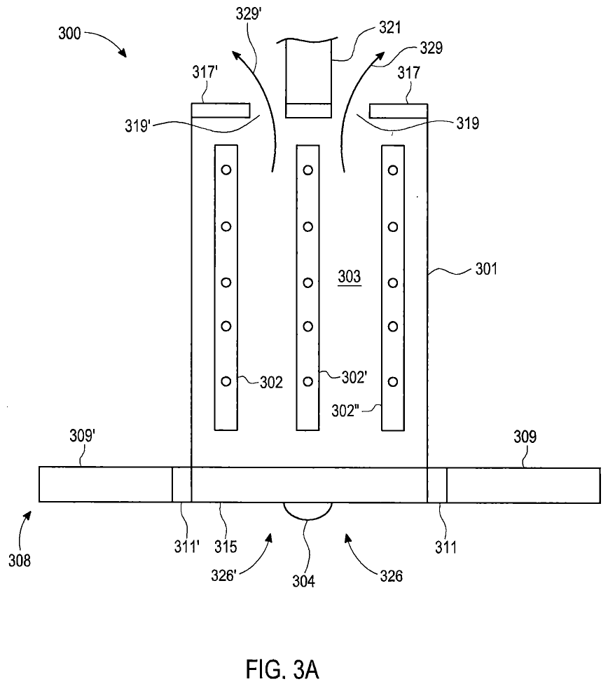

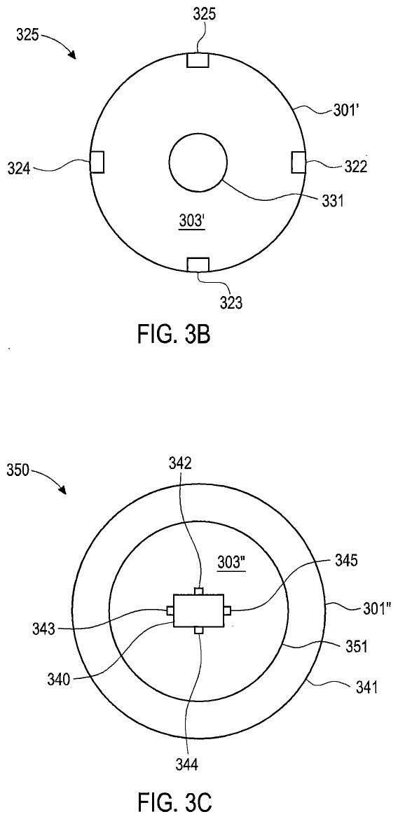

FIG. 3A shows a schematic representation of an air treatment fixture 300, such as the air treatment fixtures 241, 241′ and 241″ above. The air treatment fixture includes a germicidal air circulation unit 301, that has an internal germicidal chamber 303. Within the germicidal chamber there are UVC LED light engines 302, 302′ and 302″. The air treatment fixture 300 has a fan and filter unit 315 that is configured to draw air into the germicidal chamber 303, as indicated by the arrows 326 and 326′. With the air in drawn into the germicidal chamber 303, the air is exposed to UVC light by the UVC LED light engines 302, 302′ and 302″ and is expelled though one or more exhaust vents or exhaust ports 319 and 319′, as indicated by the arrows 329 and 329′.

The air treatment fixture 300 can have baffle structures, such as described below, within the germicidal chamber 303 and/or internal walls of the germicidal chamber are coated or made of a UV reflective materials, such as titanium oxide or zinc oxide. The air treatment fixture 300 can also include one or more sensors 317 and 317′, such as a CO₂ sensor and/or a VOC sensor. The one or more sensors 317 and 317′ are in electrical communication with a control unit, such as the control unit 103 (FIG. 1) that will regulate air flow provided by the fan and filter unit 315 through the germicidal chamber 303 and/or the UVC LED fluence or power levels of the UVC LED light engines 302, 302′ and 302″ based levels of CO2 and/or VOCs that are detected.

The air treatment fixture 300 can include a lighting unit 308. The lighting unit 308 can, for example, includes white light LED light engine 311 and 311′ that are nested against an wave-guide light plate or light plates 309 and 309′ for distributing light emitted by the white light LED light engine 311 and 311′. Preferably, the air treatment fixture 300 also includes an occupancy sensor 304 that turns on and off the lighting unit 308 and/or the any portion of the air circulation unit 301 based on occupancy detected. It will be clear to one skilled in the art that any number of different configurations of a lighting unit 308 can be employed with the air treatment fixture 300 and that the lighting unit 308 can be configures to operate independently from the germicidal air circulation unit 301.

FIGS. 3B-C show cross-sectional schematic representations of germicidal chamber configurations 325 and 350 with UVC LED light engines and baffle structures. Referring to FIG. 3B, a germicidal chamber 303′ includes a housing that forms a germicidal air circulation unit 301′, such as the germicidal air circulation unit 301 described with reference to FIG. 3A. The germicidal air circulation unit 301′ includes UVC LED light engines 322, 323, 324 and 325 positioned around the periphery of the housing of the germicidal air circulation unit 301′. The germicidal air circulation unit 301′ also includes a centrally located baffle structure 331 that helps create convention within the germicidal air circulation unit 301′ as air is drawing through the germicidal chamber 303′ by a fan and filter unit, such as the fan and filter unit 315 (FIG. 3A). When air is drawn into the germicidal chamber 303′ by a fan and filter unit, the air is exposed to the UVC LED light engines 322, 323, 324 and 325 and then the treated air is then expelled through one or more exhaust vents or exhaust ports, such as the exhaust vents or exhaust ports 319 and 319′ (FIG. 3A).

Referring to FIG. 3C, a germicidal chamber 303″ includes a housing that forms a germicidal air circulation unit 301″, such as the germicidal air circulation unit 301 described with reference to FIG. 3A. The germicidal air circulation unit 301″ includes UVC LED light engines 342, 343, 344 and 345 positioned within a center of the germicidal chamber 303″. The germicidal air circulation unit 301″ also includes a baffle structure 351 around the periphery of the housing of the germicidal air circulation unit 301″ that helps create convention within the germicidal air circulation unit 301″ as air is drawn through the germicidal chamber 303″ by a fan and filter unit, such as the fan and filter unit 315 (FIG. 3A). When air is drawn into the germicidal chamber 303″ by a fan and filter unit, the air is exposed to the UVC LED light engines 342, 343, 344 and 345 and then the treated air is then expelled through one or more exhaust vents or exhaust ports, such as the exhaust vents or exhaust ports 319 and 319′ (FIG. 3A).

While the germicidal chamber configurations 325 and 350 are shown as having round cross-sectional shapes, any number of cross-sectional shapes are envisioned including, but not limited to, square, rectangular and irregular cross-sectional shapes. Further, fans can be configured draw air into the germicidal chamber by pushing air into the germicidal chamber, pulling air into the germicidal chamber or a combination thereof depending on the position or positions of the fans on the germicidal air circulation unit.

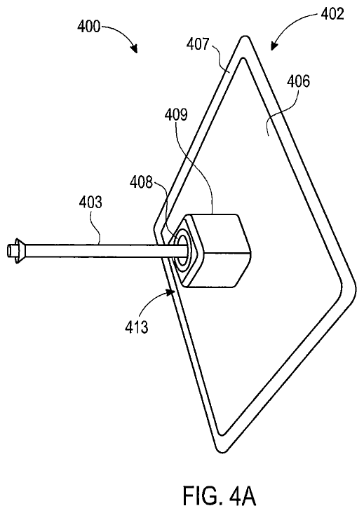

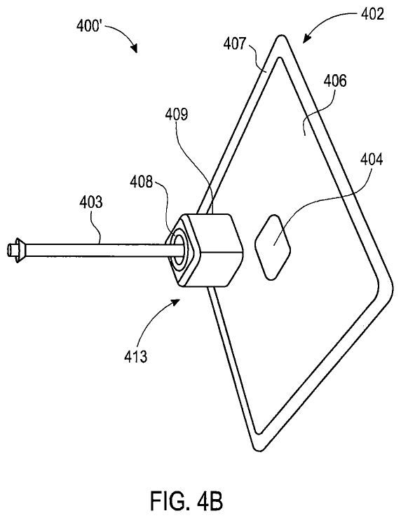

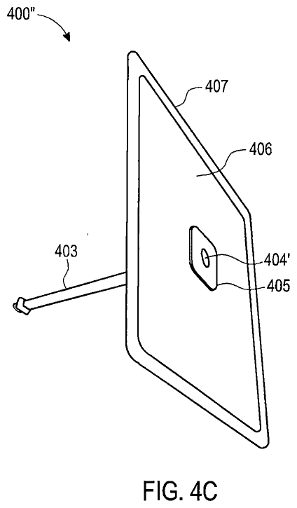

FIGS. 4A-C show views 400, 400′ and 400″ of an air treatment fixture. The air treatment fixture includes a germicidal air circulation unit 413, such as described above. The germicidal air circulation unit 413 includes a housing 409 that forms a germicidal camber. Within the germicidal chamber there are UVC LED light engines and a filter and fan unit, such as described above. The germicidal chamber can also includes baffle structures and can be coated or formed from UV reflective materials, such as zinc oxide or titanium oxide.

In operation air is drawn into the germicidal chamber of the germicidal air circulation unit 413 through an intake vent 404′ by a fan and filter unit. The air is exposed to the UVC LED light engines within the germicidal chamber and the treated air is then expelled from the germicidal chamber through an exhaust vent or exhaust port 408.

The germicidal air circulation unit 413 can also have one or more sensors, such as a CO₂ sensor and/or a VOC sensor. The one or more sensors are in electrical communication with a control unit, such as the control unit 103 (FIG. 1), wherein the control unit regulates air flow provided by the fan and filter unit and/or the fluence or power levels of the UVC LED light engines based levels of CO2 and/or VOCs that are detected by the sensors.

The air treatment fixture can includes a lighting unit 402. The lighting unit 402 includes a white light LED light engine or strip 407 that is nested against a wave-guide light plate 406 for distributing light emitted by the white light LED light engine or strip 407. Preferably, the air treatment fixture also includes an occupancy sensor that turns on and off the lighting unit 402 and/or the any portion of the air circulation unit 413 bases on occupancy detected. The lighting unit 402 can be configure to operate independently from the germicidal air circulation unit 413.

The lighting unit 402 can be configured to be removable, as shown in FIG. 4B. In operation the wave-guide light plate 406 is secured to the germicidal air circulation unit 413 through a facet plate 405 that can be detached to release the wave-guide light plate 406 from the germicidal air circulation unit 413. Preferably, the air treatment fixture also includes a mounting post 403 that can house wiring that connects to a power supply to power the air treatment fixture and allows the air treatment fixture to be suspended from a ceiling.

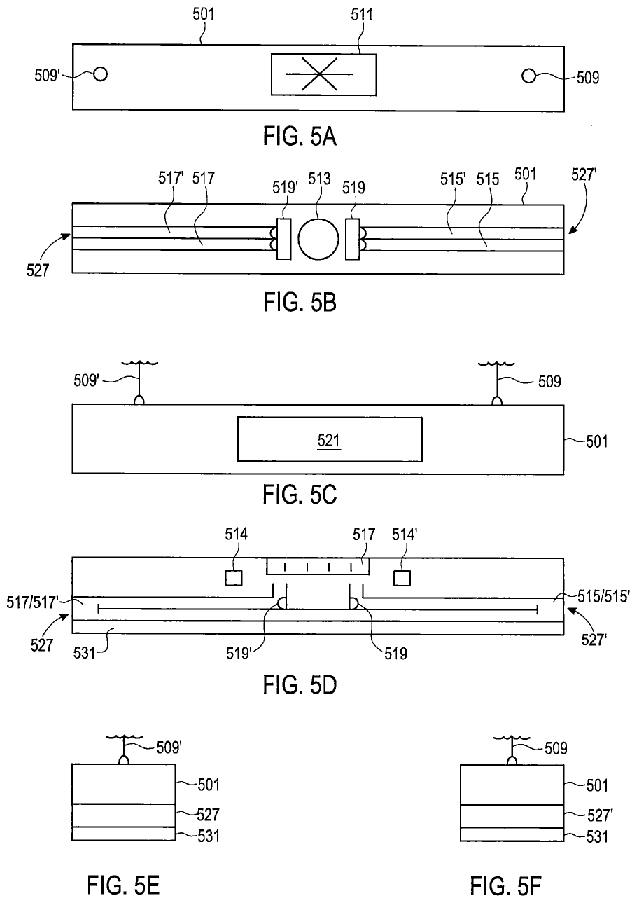

FIG. 5A-F show views of a pendent-style linear air treatment fixture with a linear germicidal air circulation unit with bi-directional sets of reflective raceways or germicidal cambers 515/515′ and 517/517′. The air treatment fixture includes a housing 501 with mounting hardware 509 and 509′ for mounting the air treatment fixture to a wall or suspending the air treatment fixture from a ceiling. On a top surface of the pendent-style linear air treatment fixture there is an exhaust port 511 coupled to fan unit 517 for drawing air into germicidal cambers 515/515′ and 517/517′ through intake ports 527 and 527′. Preferably the intake ports 527 and 527′ support filters that filter air prior to the air being drawing into the germicidal cambers 515/515′ and 517/517′ by the fan unit 517. The germicidal cambers 515/515′ and 517/517′ are elongated raceway-like structure formed from a reflective material or materials including, but not limited to polished aluminum. The germicidal cambers 515/515′ and 517/517′ can be coated with any number of UVC reflective paints or films including paints or films containing zinc oxide or titanium oxide.

Positioned at interior ends of the germicidal cambers 515/515′ and 517/517′ there are UVC LED light engines 519 and 519′ that emit UVC light and expose air within the germicidal cambers 515/515′ and 517/517′ as the air is drawn through the germicidal cambers 515/515′ and 517/517′. The housing 501 has an access panel 521 for accessing the fan unit 517, the UVC LED light engines 519 and 519′ and the germicidal cambers 515/515′ and 517/517′ for service or replacement.

The pendent-style linear air treatment fixture can also have one or more sensors 514 and 514′, such as a CO₂ sensor and/or a VOC sensor. The one or more sensors are in electrical communication with a control unit 513, wherein the control unit 513 regulates air flow provided by the fan unit 517 and the fluence or power levels of the UVC LED light engines 519 and 519′ based levels of CO₂ and/or VOCs that are detected by the one or more sensors 514 and 514′.

The pendent-style linear air treatment fixture can also includes a lighting unit 531 that can be an LED lighting unit. Preferably, the pendent-style air treatment fixture also includes an occupancy sensor that turns on and off the light unit 531 and or the any portion of the air circulation unit of the pendent-style air treatment fixture based on occupancy detected. The lighting unit 531 can also be configured to operate independently from the germicidal air circulation unit of the pendent-style linear air treatment fixture.

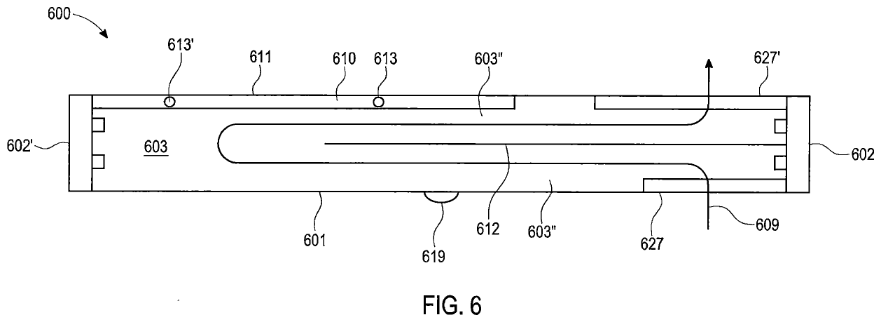

FIG. 6 shows a schematic representation of germicidal air circulation unit 600 used in air treatment fixtures, in accordance with further embodiments of the invention. The germicidal air circulation unit 600 includes an elongated housing 611 that forms an elongated and enclosed germicidal chamber 603. Within the elongated and enclosed germicidal chamber 603 there are UVC LED light engines 602 and 602′ posited at opposed ends of the elongated and enclosed germicidal chamber 603. The germicidal air circulation unit 600 also includes an intake unit 627 an exhaust unit 627′. At least one of the intake unit 627 an exhaust unit 627′ includes a fan for pulling or pushing air into the elongated and enclosed germicidal chamber 603, as indicated by the arrow 609 and at least the intake unit 627 includes a filter.

Also, within the germicidal the elongated and enclosed germicidal chamber 603 there is at least one baffle structure 612 that directs the air flow to snake through one side 603′ of the elongated and enclosed germicidal chamber 603 into and through an adjacent side 603″ of the elongated and enclosed germicidal chamber 603, as indicated by the arrow 609.

The germicidal air circulation unit 600 also includes one or more sensors 613 and 613′, such as a CO₂ sensor and/or a VOC sensor. The one or more sensors 613 and 613′ are in electrical communication with a control unit 610, such as the control unit 610 regulates air flow provided by the intake unit 627 and/or the exhaust unit 627′ and/or the fluence or power levels of the UVC LED light engines 602 and 602′ based levels of CO₂ and/or VOCs that are detected by the one or more sensors 613 and 613′. The germicidal air circulation unit 600 can also include an occupancy sensor 619 that turns on and off the germicidal air circulation unit 600 based on levels of occupancy detected. Because, the germicidal air circulation unit 600 is intended to operated when occupants are present, the occupancy sensor 619 preferably turns the germicidal air circulation unit 600 off or powers down the germicidal air circulation unit 600 when no occupants are detected and turn the germicidal air circulation unit 600 on or powers up the germicidal air circulation unit 600 when one or more occupants are detected. As described above, the germicidal air circulation unit 600 can be coupled to a lighting unit that is configured to operate in concert with or independently from the germicidal air circulation unit 600.

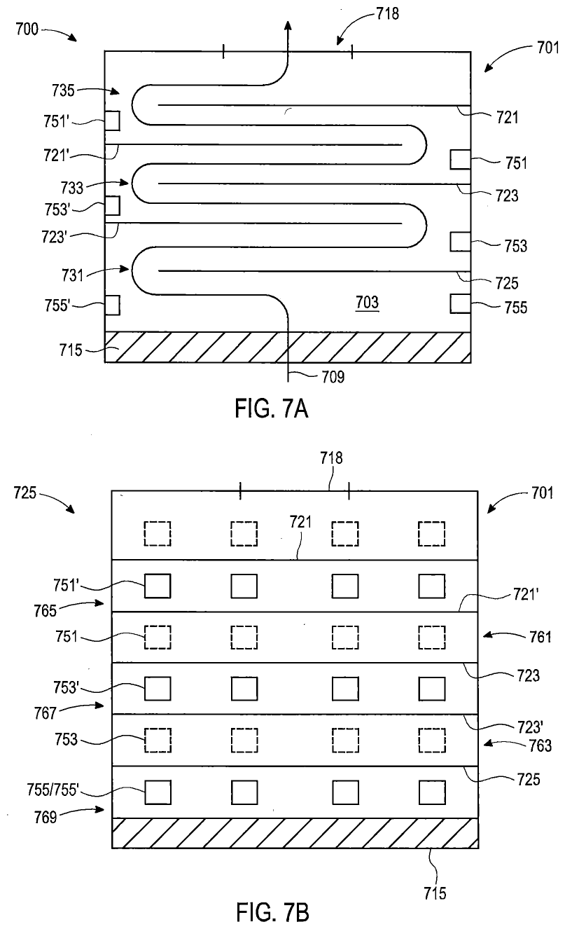

FIGS. 7A-B shows cross-sectional views 700 and 725 of a condensed germicidal air circulation unit. The condensed germicidal air circulation unit includes a housing 701 that forms an enclosed germicidal chamber 703 with stacked disinfection stages 731, 733 and 735. Within the elongated and enclosed germicidal chamber 703 there are sets UVC LED light engines 751/753/755 and 751′/753′/755′ positioned at opposed ends of the enclosed germicidal chamber 703. Each of the stacked disinfection stages 731, 733 and 735 includes at least one set of the UVC LED light engines 751/753/755 and 751′/753′/755′. The condensed germicidal air circulation unit also has baffle structures 721/723/725 and 721′/723′/725′ extending from opposed ends or sides of the enclosed germicidal chamber 703, wherein each of the stacked disinfection stages 731, 733 and 735 includes at least one baffle structures 721/723/725 and 721′/723′/725′.

The condensed germicidal air circulation unit also includes an intake unit 715 an exhaust unit 718. At least one of the intake unit 715 an exhaust unit 718 includes a fan for pulling or pushing air into enclosed germicidal chamber 703, as indicated by the arrow 709 and at least the intake unit 715 includes a filter.

In operation air is pushed or pulled into the enclosed germicidal chamber 703 and the baffle structures 721/723/725 and 721′/723′/725′ directs the air flow to snake through each of stacked disinfection stages 731, 733 and 735 where the air is exposed to the of the UVC LED light engine 751/753/755 and 751′/753′/755′ and then expelled through the exhaust unit 718.

The germicidal air circulation unit also includes one or more sensors, such as a CO₂ sensor, a VOC sensor and/or an occupancy sensor for regulating the operation of the germicidal air circulation unit based on detection of CO₂, VOCs and/or occupants by the one or more sensors. As described above, the condensed germicidal air circulation unit can be coupled to a lighting unit that is configured to operate in concert with or independently from the condensed germicidal air circulation unit and the enclosed germicidal chamber 703 and/or the baffle structures 721/723/725 and 721′/723′/725′ can be formed from or coated with a UV reflective material or materials.

Any of the germicidal cambers 303, 303′ 303″ of the germicidal air circulation units 301 (FIGS. 3A-C), the germicidal chamber of the germicidal air circulation unit 413 (FIGS. 4A-C), the germicidal cambers 515/515′ and 517/517′ of the germicidal air circulation unit (FIGS. 5A-F), the germicidal chamber 603 of the germicidal air circulation unit 600 (FIG. 6), the germicidal chamber 703 of the germicidal air circulation unit (FIGS. 7A-B) and/or baffle structures therein can be made from, contain or be coated with metal-oxide or metal-sulfide based materials. Suitable metal-oxides and metal-sulfides include, but are not limited to, Zinc Oxide (ZnO), Titanium Oxide (TiO₂), Tungsten Oxide (WO₃), Vanadium Oxide (V₂O₅), Zinc Sulfide (ZnS), and Cadmium Sulfide (CdS) and combinations thereof with or without carbon based nano-materials, such as graphene.

The present invention has been described in terms of specific embodiments incorporating details to facilitate the understanding of the principles of construction and operation of the invention. As such, references herein to specific embodiments and details thereof are not intended to limit the scope of the claims appended hereto. It will be apparent to those skilled in the art that modifications can be made in the embodiments chosen for illustration without departing from the spirit and scope of the invention.

What is claimed is:

1. An air treatment system comprising a linear germicidal air circulation unit, the linear germicidal air circulation unit comprising:
a) an elongated housing that forms a germicidal chamber;
b) bi-directional sets of reflective raceways enclosed within the germicidal chamber;
b) UVC LED light engines positioned at ends of the reflective raceways;
c) intake units with an intake ports positioned at opposed ends of the reflective raceways relative to the UVC LED light engines; and
d) an exhaust unit with a circulation fan and an exhaust port positioned between the UVC LED light engines, wherein the circulation fan pulls air into the bi-directional sets of reflective raceways through the intake units, the air is exposed to the UVC LED light engines within the reflective raceways and the air is expelled from the germicidal chamber through an exhaust port.

2. The air treatment system of claim 1, further comprising a lighting unit for providing down lighting.

3. The air treatment system of claim 1, further comprising control unit connected to one or more sensors, wherein the control unit regulates air flow provides by the circulation fan and the fluence and/or power levels of the UVC LED light engines based on levels of carbon dioxide and/or volatile organic compounds detected by the one or more sensors.

4. The air treatment system of claim 1, further comprising and occupancy sensor couple to the a control unit for turning on or off the germicidal air circulation unit and/or the lighting unit based on levels of occupancy detected by the occupancy sensor.

5. The air treatment system of claim 1, wherein portions of the germicidal chamber contain a metal-oxide material and/or a metal-sulfide material.

6. An air treatment system comprising germicidal air circulation unit, the germicidal air circulation unit comprising:
a) a housing that enclosed a UV reflective germicidal chamber with one or more baffle structures;
b) UVC LED light engines contained within the UV reflective germicidal chamber;
c) an intake unit with an intake port coupled to the UV reflective germicidal chamber;
d) an exhaust unit with an exhaust port coupled to the UV reflective germicidal chamber;
e) circulation fan that pushes or pulls air into the intake unit, through the UV reflective germicidal chamber where the air is UVC treated and then expels UVC treated air from the reflective germicidal chamber through the exhaust port.

7. The air treatment system of claim 6, wherein the housing a canister-shaped housing with one or more baffle structures attached to interior walls of the of the canister-shaped housing

8. The air treatment system of claim 7, further comprising a lighting units with a wave guided-plate that surrounds that surrounds the canister-shaped housing.

9. The air treatment system of claim 6, further comprising control unit connected to one or more sensors, wherein the control unit regulates air flow provided by the circulation fan and/or the fluence or power levels of the UVC LED light engines based on levels of carbon dioxide and/or volatile organic compounds detected by the one or more sensors.

10. The air treatment system of claim 6, further comprising and occupancy sensor couple to a control unit for turning on or off the germicidal air circulation unit and/or the lighting unit based on levels of occupancy detected by the occupancy sensor.

11. The air treatment system of claim 6, wherein portions of the UV reflective germicidal chamber contain a metal-oxide material and/or a metal-sulfide material.

12. An air treatment system comprising a condensed germicidal air circulation unit comprising:
a) a housing that forms a germicidal chamber with stacked disinfection stages in fluid communication with each other;
b) at least one least one set of the UVC LED light engines and at least one baffle structure positioned withing each of the stacked disinfection stages;
c) an intake unit with an intake port coupled to the germicidal chamber;
d) an exhaust unit with an exhaust port coupled to the coupled to the enclosed germicidal chamber; and
e) circulation fan that pushes or pulls air into the intake unit, through each of the stacked disinfection stages where the air is UVC treated in each of the stacked disinfection stages and then expels UVC treated air from the germicidal chamber through the exhaust port.

13. The air treatment system of claim 12, further comprising control unit connected to one or more sensors, wherein the control unit regulates air flow provided by the circulation fan and/or the fluence or power levels of the UVC LED light engines based on levels of carbon dioxide and/or volatile organic compounds detected by the one or more sensors.

14. The air treatment system of claim 12, further comprising and occupancy sensor couple to a control unit for turning on or off the condensed germicidal air circulation unit based on levels of occupancy detected by the occupancy sensor.

15. The air treatment system of claim 1, wherein portions of the germicidal chamber contain a metal-oxide material and/or a metal-sulfide material.
